(19) United States
(12) Patent Application Publication
(10) Pub. No.: US 2002/0058277 A1
(43) Pub. Date: May 16, 2002

(54) Nucleotide sequences which code for the Gap2 protein

(75) Inventors: Brigitte Bathe, Salzkotten (DE); Stephan Hans, Osnabrueck (DE); Walter Pfefferle, Halle (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/948,619

(22) Filed: Sep. 10, 2001

(30) Foreign Application Priority Data
Sep. 9, 2000 (DE) 100 44 754.6
Jul. 28, 2001 (DE) 101 36 985.9

(51) Int. Cl. C12Q 1/68; C07H 21/04; C12P 19/34; C12N 9/02; C12N 1/21

(52) U.S. Cl. 435/6; 435/189; 435/320.1; 435/252.3; 435/91.1; 536/23.2

(57) ABSTRACT

The present invention provides nucleotide sequences from Coryneform bacteria which code for the Gap2 protein and a process for the fermentative preparation of amino acids using bacteria in which the gap2 gene is enhanced.

### CROSS-REFERENCE TO RELATED APPLICATIONS

The present application claims priority to German Application No. DE 10044754.6, which was filed on Sep. 9, 2000 and German Application No. DE 10136985.9, which was filed on Jul. 28, 2001; the entire contents of both documents are incorporated herein by reference.

### BACKGROUND OF THE INVENTION

1. Field of the Invention

The present invention provides nucleotide sequences from Coryneform bacteria which code for the Gap2 protein and a process for the fermentative preparation of amino acids using bacteria in which the gap2 gene is enhanced.

2. Discussion of the Background

L-Amino acids, in particular L-lysine, are used in human medicine and in the pharmaceuticals industry, in the foodstuffs industry and especially in animal nutrition.

It is known that amino acids are prepared by fermentation from strains of Coryneform bacteria, in particular Corynebacterium glutamicum. Because of their great importance, work is constantly being undertaken to improve the processes for preparing amino acids. Improvements to the process can relate to fermentation measures, such as stirring and supply of oxygen; the composition of the nutrient media, such as adjusting the sugar concentration during the fermentation; working up the product form by, for example, ion exchange chromatography; or by altering the intrinsic output properties of the microorganism itself.

Methods of mutagenesis, selection and mutant selection are used to improve the output properties of these microorganisms. Strains that are resistant to antimetabolites or are auxotrophic for metabolites of regulatory importance and produce amino acids may be obtained in this manner.

Methods of the recombinant DNA technique have also been employed for some years for improving the strain of Corynebacterium strains which produce L-amino acids, by amplifying individual amino acid biosynthesis genes and investigating the effect on the amino acid production.

However, there remains a critical need for improved methods of producing L-amino acids and thus for the provision of strains of bacteria producing higher amounts of L-amino acids. On a commercial or industrial scale even small improvements in the yield of L-amino acids, or the efficiency of their production, are economically significant. Prior to the present invention, it was not recognized that enhancing the gap2 gene encoding the glyceraldehydes 3-phosphate dehydrogenase 2 (Gap2) protein would improve L-amino acid yields.

### SUMMARY OF THE INVENTION

An object of the present invention is to provide novel measures for the improved production of L-amino acids or amino acid, where these amino acids include L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan, L-arginine and the salts (monohydrochloride or sulfate) thereof.

One object of the present invention is providing a novel process for improving the fermentative production of said L-amino acids, particularly L-lysine. Such a process includes enhanced bacteria, preferably enhanced Coryneform bacteria, which express enhanced amounts Gap2 glyceraldhyde 3-phosphate dehydrogenase 2 protein or protein that has glyceraldhyde 3-phosphate dehydrogenase 2 activity.

Thus, another object of the present invention is providing such a bacterium, which expresses an enhanced amount of Gap2 protein or gene products of the gap2 gene.

Another object of the present invention is providing a bacterium, preferably a Coryneform bacterium, which expresses a polypeptide that has an enhanced glyceraldhyde 3-phosphate dehydrogenase 2 activity.

Another object of the invention is to provide a nucleotide sequence encoding a polypeptide having the glyceraldhyde 3-phosphate dehydrogenase 2 (Gap2) protein sequence. One embodiment of such a sequence is the nucleotide sequence of SEQ ID NO: 1.

A further object of the invention is a method of making protein or an isolated polypeptide having glyceraldhyde 3-phosphate dehydrogenase 2 activity, as well as use of such isolated polypeptides in the production of amino acids. One embodiment of such a polypeptide is the polypeptide having the amino acid sequence of SEQ ID NO: 2.

Other objects of the invention include methods of detecting nucleic acid sequences homologous to SEQ ID NO: 1, particularly nucleic acid sequences encoding polypeptides that have glyceraldhyde 3-phosphate dehydrogenase 2 activity, and methods of making nucleic acids encoding such polypeptides.

The above objects highlight certain aspects of the invention. Additional objects, aspects and embodiments of the invention are found in the following detailed description of the invention.

### BRIEF DESCRIPTION OF THE DRAWINGS

FIG. 1: Map of the plasmid pEC-K18mob2.

FIG. 2: Map of the plasmid pEC-K18mob2gap2exp.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Cold Spring Harbor Laboratory, New York (1989), Current Protocols in Molecular Biology, Ausebel et al (eds), John Wiley and Sons, Inc. New York (2000)and the various references cited therein.

“L-amino acids” or “amino acids” as used herein mean one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Lysine is particularly preferred.

Where L-lysine or lysine are mentioned in the following, this means not only the bases but also the salts, such as e.g. lysine monohydrochloride or lysine sulfate.

The invention provides an isolated polynucleotide from Coryneform bacteria, comprising a polynucleotide sequence which codes for the gap2 gene, chosen from the group consisting of

a) polynucleotide which is identical to the extent of at least 70% to a polynucleotide which codes for a polypeptide which comprises the amino acid sequence of SEQ ID No. 2,

b) polynucleotide which codes for a polypeptide which comprises an amino acid sequence which is identical to the extent of at least 70% to the amino acid sequence of SEQ ID No.2

c) polynucleotide which is complementary to the polynucleotides of a) or b), and

d) polynucleotide comprising at least 15 successive nucleotides of the polynucleotide sequence of a), b) or c),

the polypeptide preferably having the activity of glyceraldehyde 3-phosphate dehydrogenase 2.

The invention also provides the above-mentioned polynucleotide, this preferably being a DNA which is capable of replication, comprising:

(i) the nucleotide sequence shown in SEQ ID No. 1, or

(ii) at least one sequence which corresponds to sequence (i) within the range of the degeneration of the genetic code, or

(iii) at least one sequence which hybridizes with the sequence complementary to sequence (i) or (ii), and optionally

(iv) sense mutations of neutral function in (i).

The invention also provides

A polynucleotide, in particular DNA, which is capable of replication and comprises the nucleotide sequence as shown in SEQ ID No. 1;

a polynucleotide which codes for a polypeptide which comprises the amino acid sequence as shown in SEQ ID No. 2;

a vector containing the polynucleotide according to the invention, in particular a shuttle vector or plasmid vector, and

Coryneform bacteria which contain the vector or in which the endogenous gap2 gene is enhanced.

The invention also provides polynucleotides which substantially comprise a polynucleotide sequence, which are obtainable by screening by means of hybridization of a corresponding gene library of a Coryneform bacterium, which comprises the complete gene or parts thereof, with a probe which comprises the sequence of the polynucleotides according to the invention according to SEQ ID No. 1 or a fragment thereof, and isolation of the polynucleotide sequence mentioned.

Polynucleotides which comprise the sequences according to the invention are suitable as hybridization probes for RNA, cDNA and DNA, in order to isolate, in the full length, nucleic acids or polynucleotides or genes which code for glyceraldehyde 3-phosphate dehydrogenase 2 or to isolate those nucleic acids or polynucleotides or genes which have a high similarity of sequence with that of the gap2 gene. They are also suitable for incorporation into so-called “arrays”, “micro arrays” or “DNA chips” in order to detect and to determine the corresponding polynucleotides.

Polynucleotides which comprise the sequences according to the invention are furthermore suitable as primers with the aid of which DNA of genes which code for glyceraldehyde 3-phosphate dehydrogenase 2 can be prepared by the polymerase chain reaction (PCR).

Such oligonucleotides which serve as probes or primers comprise at least 25, 26, 27, 28, 29 or 30, preferably at least 20, 21, 22, 23 or 24, very particularly preferably at least 15, 16, 17, 18 or 19 successive nucleotides. Oligonucleotides which have a length of at least 31, 32, 33, 34, 35, 36, 37, 38, 39 or 40, or at least 41, 42, 43, 44, 45, 46, 47, 48, 49 or 50 nucleotides are also suitable. Oligonucleotides with a length of at least 100, 150, 200, 250 or 300 nucleotides are optionally also suitable.

“Isolated” means separated out of its natural environment.

“Polynucleotide” in general relates to polyribonucleotides and polydeoxyribonucleotides, it being possible for these to be non-modified RNA or DNA or modified RNA and DNA.

The polynucleotides according to the invention include a polynucleotide according to SEQ ID No. 1 or a fragment prepared therefrom and also those which are at least in particular 70% to 80%, preferably at least 81% to 85%, particularly preferably at least 86% to 90%, and very particularly preferably at least 91%, 93%, 95%, 97% or 99% identical to the polynucleotide according to SEQ ID No. 1 or a fragment prepared therefrom.

“Polypeptides” are understood as meaning peptides or proteins which comprise amino acids bonded via two or more peptide bonds.

The polypeptides according to the invention include a polypeptide according to SEQ ID No. 2, in particular those with the biological activity of glyceraldehyde 3-phosphate dehydrogenase 2 and also those which are at least 70% to 80%, preferably at least 81% to 85%, particularly preferably at least 86% to 90%, and very particularly preferably at least 91%, 93%, 95%, 97% or 99% identical to the polypeptide according to SEQ ID No. 2 and have the activity mentioned.

The invention furthermore relates to a process for the fermentative preparation of amino acids chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using Coryneform bacteria which in particular already produce amino acids and in which the nucleotide sequences which code for the gap2 gene are enhanced, in particular over-expressed.

The term “enhancement” in this connection describes the increase in the intracellular activity of one or more enzymes in a microorganism which are coded by the corresponding DNA, for example by increasing the number of copies of the gene or genes, using a potent promoter or using a gene which codes for a corresponding enzyme having a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

Preferably, a bacterial strain with enhanced expression of a gap2 gene that encodes a polypeptide with glyceraldehydes 3-phosphate dehydrogenase 2 activity will improve amino acid yield at least 1%.

The microorganisms which the present invention provides can produce L-amino acids from glucose, sucrose, lactose, fructose, maltose, molasses, starch, cellulose or from glycerol and ethanol. They can be representatives of Coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular the species Corynebacterium glutamicum (C. glutamicum), are in particular the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

and L-amino acid-producing mutants or strains prepared therefrom.

The new gap2 gene from C. glutamicum which codes for the enzyme glyceraldehyde 3-phosphate dehydrogenase 2 (EC 1.2.1.12) has been isolated.

To isolate the gap2 gene or also other genes of C. glutamicum, a gene library of this microorganism is first set up in Escherichia coli (E. coli). The setting up of gene libraries is described in generally known textbooks and handbooks. The textbook by Winnacker: Gene und Klone, Eine Einführung in die Gentechnologie (Verlag Chemie, Weinheim, Germany, 1990), or the handbook by Sambrook et al.: Molecular Cloning, A Laboratory Manual (Cold Spring Harbor Laboratory Press, 1989) may be mentioned as an example. A well-known gene library is that of the E. coli K-12 strain W3110 set up in λ vectors by Kohara et al. (Cell 50, 495-508 (1987)). Bathe et al. (Molecular and General Genetics, 252:255-265, 1996) describe a gene library of C. glutamicum ATCC13032, which was set up with the aid of the cosmid vector SuperCos I (Wahl et al., 1987, Proceedings of the National Academy of Sciences, USA, 84:2160-2164) in the E. coli K-12 strain NM554 (Raleigh et al., 1988, Nucleic Acids Research 16:1563-1575).

Börmann et al. (Molecular Microbiology 6(3), 317-326)) (1992)) in turn describe a gene library of C. glutamicum ATCC13032 using the cosmid pHC79 (Hohn and Collins, Gene 11, 291-298 (1980)).

To prepare a gene library of C. glutamicum in E. coli it is also possible to use plasmids such as pBR322 (Bolivar, Life Sciences, 25, 807-818 (1979)) or pUC9 (Vieira et al., 1982, Gene, 19:259-268). Suitable hosts are, in particular, those E. coli strains which are restriction- and recombination-defective. An example of these is the strain DH5αmcr, which has been described by Grant et al. (Proceedings of the National Academy of Sciences USA, 87 (1990) 4645-4649). The long DNA fragments cloned with the aid of cosmids can in turn be subcloned in the usual vectors suitable for sequencing and then sequenced, as is described e.g. by Sanger et al. (Proceedings of the National Academy of Sciences of the United States of America, 74:5463-5467, 1977).

The DNA sequences obtained may then be examined using known algorithms or sequence analysis programs such as e.g. the one from Staden (Nucleic Acids Research 14, 217-232(1986)), the one from Marck (Nucleic Acids Research 16, 1829-1836 (1988)) or the GCG program from Butler (Methods of Biochemical Analysis 39, 74-97 (1998)).

Additionally, methods employing DNA chips, microarrays or similar recombinant DNA technology that enables high throughput screening of DNA and polynucleotides which encode the glyceraldehydes 3-phosphate dehydrogenase 2 protein or polynucleotides with homology to the gap2 gene as described herein. Such methods are known in the art and are described, for example, in Current Protocols in Molecular Biology, Ausebel et al (eds), John Wiley and Sons, Inc. New York (2000).

The new DNA sequence of C. glutamicum which codes for the gap2 gene and which, as SEQ ID No. 1, is a constituent of the present invention has been found. The amino acid sequence of the corresponding protein has furthermore been derived from the present DNA sequence by the methods described above. The resulting amino acid sequence of the gap2 gene product is shown in SEQ ID No. 2. It is known that enzymes endogenous to the host can split off the N-terminal amino acid methionine or formylmethionine of the protein formed.

Coding DNA sequences which result from SEQ ID No. 1 by the degeneracy of the genetic code are also a constituent of the invention. In the same way, DNA sequences which hybridize with SEQ ID No. 1 or parts of SEQ ID No. 1 are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. are of neutral function. Such mutations are also called, inter alia, neutral substitutions. It is furthermore known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences which result in a corresponding manner from SEQ ID No. 2 are also a constituent of the invention.

In the same way, DNA sequences which hybridize with SEQ ID No. 1 or parts of SEQ ID No. 1 are a constituent of the invention. Finally, DNA sequences which are prepared by the polymerase chain reaction (PCR) using primers which result from SEQ ID No. 1 are a constituent of the invention. Such oligonucleotides typically have a length of at least 15 nucleotides.

Instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (International Journal of Systematic Bacteriology (1991) 41: 255-260). The hybridization takes place under stringent conditions, that is to say only hybrids in which the probe and target sequences, i.e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5×SSC buffer at a temperature of approx. 50-68° C., for example, can be employed for the hybridization reaction. Probes can also hybridize here with polynucleotides which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) a temperature of approx. 50-68° C. being established. It is optionally possible to lower the salt concentration to 0.1×SSC. Polynucleotide fragments which are, for example, at least 70% or at least 80% or at least 90% to 95% identical to the sequence of the probe employed can be isolated by increasing the hybridization temperature stepwise from 50 to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

Instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

It has been found that Coryneform bacteria produce amino acids in an improved manner after over-expression of the gap2 gene.

To achieve an over-expression, the number of copies of the corresponding genes can be increased, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative amino acid production. The expression is likewise improved by measures to prolong the life of the m-RNA. Furthermore, the enzyme activity is also increased by preventing the degradation of the enzyme protein. The genes or gene constructs can either be present in plasmids with a varying number of copies, or can be integrated and amplified in the chromosome. Alternatively, an over-expression of the genes in question can furthermore be achieved by changing the composition of the media and the culture procedure.

Instructions in this context can be found by the expert, inter alia, in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European Patent Specification 0 472 869, in U.S. Pat. No. 4,601,893, in Schwarzer and Puhler (Bio/Technology 9, 84-87 (1991)), Reinscheid et al. (Applied and Environmental Microbiology 60, 126-132 (1994)), in LaBarre et al. (Journal of Bacteriology 175, 1001-1007 (1993)), in Patent Application WO 96/15246, in Malumbres et al. (Gene 134, 15-24 (1993)), in Japanese Laid-Open Specification JP-A-10-229891, in Jensen and Hammer (Biotechnology and Bioengineering 58, 191-195 (1998)), in Makrides (Microbiological Reviews 60:512-538 (1996)) and in known textbooks of genetics and molecular biology.

By way of example, for enhancement the gap2 gene according to the invention was over-expressed with the aid of episomal plasmids. Suitable plasmids are those which are replicated in Coryneform bacteria. Numerous known plasmid vectors, such as e.g. pZ1 (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)) are based on the cryptic plasmids pHM1519, pBL1 or pGA1. Other plasmid vectors, such as e.g. those based on pCG4 (U.S. Pat. No. 4,489,160), or pNG2 (Serwold-Davis et al., FEMS Microbiology Letters 66, 119-124 (1990)), or pAGl (U.S. Pat. No. 5,158,891), can be used in the same manner.

Plasmid vectors which are furthermore suitable are also those with the aid of which the process of gene amplification by integration into the chromosome can be used, as has been described, for example, by Reinscheid et al. (Applied and Environmental Microbiology 60, 126-132 (1994)) for duplication or amplification of the hom-thrB operon. In this method, the complete gene is cloned in a plasmid vector which can replicate in a host (typically E. coli), but not in C. glutamicum. Possible vectors are, for example, pSUP301 (Simon et al., Bio/technology 1, 784-791 (1983)), pK18mob or pK19mob (Schäfer et al., Gene 145, 69-73 (1994)), pGEM-T (Promega corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)), pEM1 (Schrumpf et al., 1991, Journal of Bacteriology 173:4510-4516) or pBGS8 (Spratt et al.,1986, Gene 41: 337-342). The plasmid vector which contains the gene to be amplified is then transferred into the desired strain of C. glutamicum by conjugation or transformation. The method of conjugation is described, for example, by Schäfer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross over” event, the resulting strain contains at least two copies of the gene in question. In addition, it may be advantageous for the production of L-amino acids to enhance, in particular over-express one or more enzymes of the particular biosynthesis pathway, of glycolysis, of anaplerosis, of the citric acid cycle, of the pentose phosphate cycle, of amino acid export and optionally regulatory proteins, in addition to the gap2 gene.

Thus, for example, for the preparation of L-amino acids, in addition to enhancement of the gap2 gene, one or more endogenous genes chosen from the group consisting of

the dapA gene which codes for dihydrodipicolinate synthase (EP-B 0 197 335),

the gap gene which codes for glyceraldehyde 3-phosphate dehydrogenase (Eikmanns (1992), Journal of Bacteriology 174:6076-6086),

the tpi gene which codes for triose phosphate isomerase (Eikmanns (1992), Journal of Bacteriology 174:6076-6086),

the pgk gene which codes for 3-phosphoglycerate kinase (Eikmanns (1992), Journal of Bacteriology 174:6076-6086),

the zwf gene which codes for glucose 6-phosphate dehydrogenase (JP-A-09224661),

the pyc gene which codes for pyruvate carboxylase (DE-A 198 31 609),

the mqo gene which codes for malate-quinone oxidoreductase (Molenaar et al., European Journal of Biochemistry 254, 395-403 (1998)),

the lysC gene which codes for a feed-back resistant aspartate kinase (Accession No. P26512; EP-B-0387527; EP-A-0699759),

the lysE gene which codes for lysine export (DE-A-195 48 222),

the hom gene which codes for homoserine dehydrogenase (EP-A 0131171),

the ilvA gene which codes for threonine dehydratase (Möckel et al., Journal of Bacteriology (1992) 8065-8072) or the ilvA(Fbr) allele which codes for a feed back resistant threonine dehydratase (Möckel et al., (1994) Molecular Microbiology 13: 833-842),

the ilvBN gene which codes for acetohydroxy-acid synthase (EP-B 0356739),

the ilvD gene which codes for dihydroxy-acid dehydratase (Sahm and Eggeling (1999) Applied and Environmental Microbiology 65: 1973-1979),

the zwa1 gene which codes for the Zwa1 protein (DE: 19959328.0, DSM 13115)

can be enhanced, in particular over-expressed.

It may furthermore be advantageous for the production of L-amino acids, in addition to the enhancement of the qap2 gene, for one or more genes chosen from the group consisting of:

the pck gene which codes for phosphoenol pyruvate carboxykinase (DE 199 50 409.1; DSM 13047),

the pgi gene which codes for glucose 6-phosphate isomerase (US 09/396,478; DSM 12969),

the poxB gene which codes for pyruvate oxidase (DE: 1995 1975.7; DSM 13114),

the zwa2 gene which codes for the Zwa2 protein (DE: 19959327.2, DSM 13113)

to be attenuated, in particular for the expression thereof to be reduced.

The term “attenuation” in this connection describes the reduction or elimination of the intracellular activity of one or more enzymes (proteins) in a microorganism which are coded by the corresponding DNA, for example by using a weak promoter or using a gene or allele which codes for a corresponding enzyme with a low activity or inactivates the corresponding gene or enzyme (protein), and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

In addition to over-expression of the gap2 gene it may furthermore be advantageous for the production of amino acids to eliminate undesirable side reactions (Nakayama: “Breeding of Aminoacid Producing Micro-organisms”, in: Overproduction of Microbial Products, Krumphanzl, Sikyta, Vanek (eds.), Academic Press, London, UK, 1982).

The invention also provides the microorganisms prepared according to the invention, and these can be cultured continuously or discontinuously in the batch process (batch culture) or in the fed batch (feed process) or repeated fed batch process (repetitive feed process) for the purpose of production of amino acids. A summary of known culture methods is described in the textbook by Chmiel (Bioprozesstechnik 1. Einführung in die Bioverfahrenstechnik (Gustav Fischer Verlag, Stuttgart, 1984)) or in the textbook by Storhas (Bioreaktoren und periphere Einrichtungen (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e. g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C. and preferably 25° C. to 40° C. Culturing is continued until a maximum of the desired product has formed. This target is usually reached within 10 hours to 160 hours.

Methods for the determination of L-amino acids are known from the prior art. The analysis can thus be carried out, for example, as described by Spackman et al. (Analytical Chemistry, 30, (1958), 1190) by ion exchange chromatography with subsequent ninhydrin derivation, or it can be carried out by reversed phase HPLC, for example as described by Lindroth et al. (Analytical Chemistry (1979) 51: 1167-1174).

The process according to the invention is used for fermentative preparation of amino acids.

The following microorganism was deposited as a pure culture on Jul. 18, 2001 at the Deutsche Sammlung für Mikroorganismen und Zellkulturen (DSMZ=German Collection of Microorganisms and Cell Cultures, Braunschweig, Germany) in accordance with the Budapest Treaty:

Escherichia coli DH5αmcr/pEC-K18mob2gap2exp as DSM 14407.

The present invention is explained in more detail in the following with the aid of embodiment examples.

The isolation of plasmid DNA from Escherichia coli and all techniques of restriction, Klenow and alkaline phosphatase treatment were carried out by the method of Sambrook et al. (Molecular Cloning. A Laboratory Manual (1989) Cold Spring Harbor Laboratory Press, Cold Spring Harbor, N.Y., USA). Methods for transformation of Escherichia coli are also described in this handbook.

The composition of the usual nutrient media, such as LB or TY medium, can also be found in the handbook by Sambrook et al.

Having generally described this invention, a further understanding can be obtained by reference to certain specific examples which are provided herein for purposes of illustration only, and are not intended to be limiting unless otherwise specified.

### EXAMPLES

### Example 1

Preparation of a Genomic Cosmid Gene Library from Corynebacterium glutamicum ATCC 13032

Chromosomal DNA from Corynebacterium glutamicum ATCC 13032 was isolated as described by Tauch et al. (1995), Plasmid 33:168-179) and partly cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Diagnostics GmbH, Mannheim, Germany, Product Description SAP, Code No. 1758250). The DNA of the cosmid vector SuperCos1 (Wahl et al. (1987) Proceedings of the National Academy of Sciences USA 84:2160-2164), obtained from Stratagene (La Jolla, USA, Product Description SuperCos1 Cosmid Vector Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

Finally, the cosmid DNA was cleaved with the restriction enzyme BamHI (Amersham Pharmacia, Freiburg, Germany, Product Description BamHI, Code no. 27-0868-04). The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no. 27-0870-04). The ligation mixture was then packed in phages with the aid of Gigapack II XL Packing Extract (Stratagene, La Jolla, USA, Product Description Gigapack II XL Packing Extract, Code no. 200217).

For infection of the E. coli strain NM554 (Raleigh et al. 1988, Nucleic Acid Research 16:1563-1575) the cells were taken up in 10 mM MgSO₄ and mixed with an aliquot of the phage suspension. The infection and titering of the cosmid library were carried out as described by Sambrook et al. (1989, Molecular Cloning: A laboratory Manual, Cold Spring Harbor), the cells being plated out on LB agar (Lennox, 1955, Virology, 1:190) with 100 mg/l ampicillin. After incubation overnight at 37° C., recombinant individual clones were selected.

### Example 2

Isolation and Sequencing of the gap2 Gene

The cosmid DNA of an individual colony was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product-No. 27-0913-02). The DNA fragments were dephosphorylated with shrimp alkaline phosphatase (Roche Diagnostics GmbH, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany).

The DNA of the sequencing vector pZero-1, obtained from Invitrogen (Groningen, The Netherlands, Product Description Zero Background Cloning Kit, Product No. K2500-01), was cleaved with the restriction enzyme BamHI (Amersham Pharmacia, Freiburg, Germany, Product Description BamHI No. 27-0868-04). The ligation of the cosmid fragments in the sequencing vector pZero-1 was carried out as described by Sambrook et al. (1989, Molecular Cloning: A Laboratory Manual, Cold Spring Harbor), the DNA mixture being incubated overnight with T4 ligase (Pharmacia Biotech, Freiburg Germany). This ligation mixture was then electroporated (Tauch et al. 1994, FEMS Microbiol Letters, 123:343-7) into the E. coli strain DH5αMCR (Grant, 1990, Proceedings of the National Academy of Sciences U.S.A., 87:4645-4649) and plated out on LB agar (Lennox, 1955, Virology 1:190) with 50 mg/l zeocin.

The plasmid preparation of the recombinant clones was carried out with the Biorobot 9600 (Product No. 900200, Qiagen, Hilden, Germany). The sequencing was carried out by the dideoxy chain termination method of Sanger et al. (1977, Proceedings of the National Academy of Sciences U.S.A., 74:5463-5467) with modifications according to Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. The separation by gel electrophoresis and analysis of the sequencing reaction were carried out in a “Rotiphoresis NF Acrylamide/Bisacrylamidel” Gel (29:1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231) version 97-0. The individual sequences of the pZerol derivatives were assembled to a continuous contig. The computer-assisted coding region analysis was prepared with the XNIP program (Staden, 1986, Nucleic Acids Research, 14:217-231).

The resulting nucleotide sequence is shown in SEQ ID No. 1. Analysis of the nucleotide sequence showed an open reading frame of 1440 base pairs, which was called the gap2 gene. The gap2 gene codes for a protein of 480 amino acids.

### Example 3

Preparation of a Shuttle Vector pEC-K18mob2gap2exp for Enhancement of the gap2 Gene in C. glutamicum

3.1 Cloning of the gap2 Gene in the Vector pCR®Blunt II

From the strain ATCC 13032, chromosomal DNA was isolated by the method of Eikmanns et al. (Microbiology 140: 1817-1828 (1994)). On the basis of the sequence of the gap2 gene known for C. glutamicum from example 2, the following oligonucleotides were chosen for the polymerase chain reaction (see also SEQ ID No. 3 and SEQ ID No. 4):

gap2exp1:

5′-TTG AAG TGG AGC CGG ACG AG-3′

gap2exp2:

5′-CCT ATA GTA CGG CTG GCT GC-3′

The primers shown were synthesized by ARK Scientific GmbH Biosystems (Darmstadt, Germany) and the PCR reaction was carried out by the standard PCR method of Innis et al. (PCR protocols. A Guide to Methods and Applications, 1990, Academic Press) with Pwo-Polymerase from Roche Diagnostics GmbH (Mannheim, Germany). With the aid of the polymerase chain reaction, the primers allow amplification of a DNA fragment 2022 bp in size, which carries the gap2 gene with the potential promoter region. The DNA sequence of the amplified DNA fragment was checked by sequencing.

The amplified DNA fragment was ligated with the Zero Blunt™ Kit of Invitrogen Corporation (Carlsbad, Calif., USA; Catalogue Number K2700-20) in den vector pCR®Blunt II (Bernard et al., Journal of Molecular Biology, 234: 534-541 (1993)).

The E. coli strain TOP10 was then electroporated with the ligation batch (Hanahan, In: DNA cloning. A Practical Approach. Vol. I, IRL-Press, Oxford, Washington D.C., USA, 1985). Selection for plasmid-carrying cells was made by plating out the transformation batch on LB agar (Sambrook et al., Molecular Cloning: A Laboratory Manual. 2^{nd} Ed., Cold Spring Harbor Laboratory Press, Cold Spring Harbor, N.Y., 1989), which had been supplemented with 25 mg/l kanamycin. Plasmid DNA was isolated from a transformant with the aid of the QIAprep Spin Miniprep Kit from Qiagen and checked by restriction with the restriction enzyme EcoRI and subsequent agarose gel electrophoresis (0.8%). The plasmid was called pCRB1-gap2exp.

3.2 Preparation of the E. coli—C. glutamicum Shuttle Vector pEC-K18mob2

The E. coli—C. glutamicum shuttle vector was constructed according to the prior art. The vector contains the replication region rep of the plasmid pGA1 including the replication effector per (U.S. Pat. No. 5,175,108; Nesvera et al., Journal of Bacteriology 179, 1525-1532 (1997)), the kanamycin resistance-imparting aph(3′)-IIa gene of the transposon Tn5 (Beck et al., Gene 19, 327-336 (1982)), the replication region oriV of the plasmid pMB1 (Sutcliffe, Cold Spring Harbor Symposium on Quantitative Biology 43, 77-90 (1979)), the lacZα gene fragment including the lac promoter and a multiple cloning site (mcs) (Norrander, J. M. et al., Gene 26, 101-106 (1983)) and the mob region of the plasmid RP4 (Simon et al., Bio/Technology 1:784-791 (1983)).

The vector pEC-K18mob2 constructed was transferred into C. glutamicum DSM5715 by means of electroporation (Liebl et al., 1989, FEMS Microbiology Letters, 53:299-303). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 25 mg/l kanamycin. Incubation was carried out for 2 days at 33° C.

Plasmid DNA was isolated from a transformant by conventional methods (Peters-Wendisch et al., 1998, Microbiology, 144, 915 -927), cleaved with the restriction endonucleases EcoRI and HindIII, and the plasmid was checked by subsequent agarose gel electrophoresis.

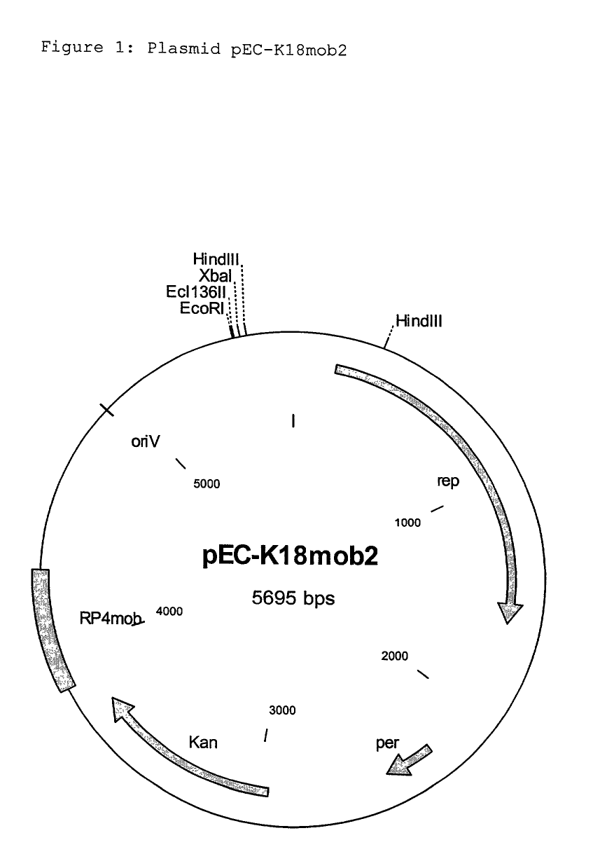

The plasmid construction thus obtained was called pEC-K18mob2 and is shown in FIG. 1. The strain obtained by electroporation of the plasmid pEC-K18mob2 in the C. glutamicum strain DSM5715 was called DSM5715/pEC-K18mob2 and deposited on Jan. 20, 2000 as DSM13245 at the Deutsche Sammlung für Mikroorganismen und Zellkulturen (DSMZ=German Collection of Microorganisms and Cell Cultures, Braunschweig, Germany) in accordance with the Budapest Treaty.

3.3 Cloning of gap2 in the E. coli—C. glutamicum Shuttle Vector pEC-K18mob2

For cloning of the gap2 gene in the E. coli—C. glutamicum shuttle vector pEC-K18mob2 described in example 3.2, plasmid DNA of pEC-K18mob2 was cleaved completely with the restriction endonucleases Ecl136II and XbaI and treated with alkaline phosphatase (Alkaline Phosphatase, Roche Diagnostics GmbH, Mannheim, Germany).

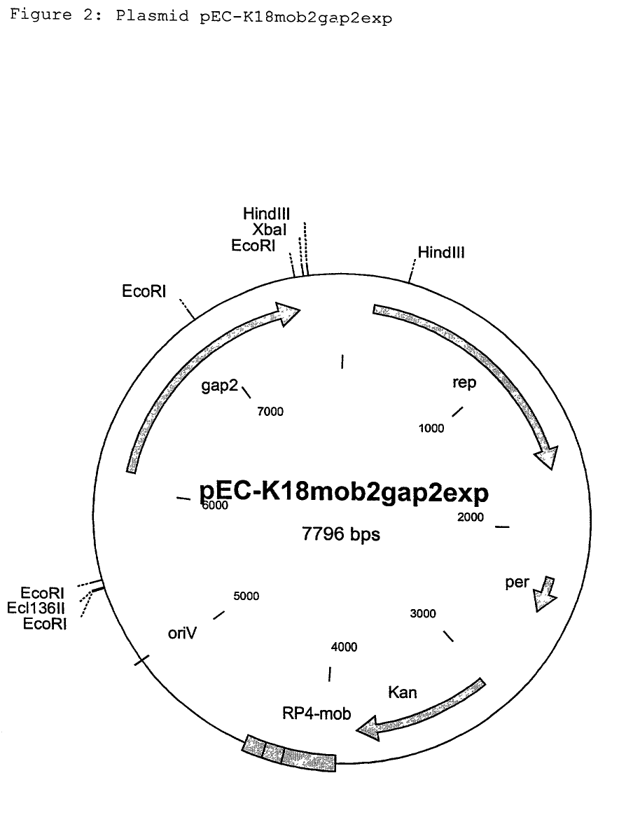

The vector pCRBl-gap2exp was isolated from Escherichia coli Top10 and cleaved completely with the restriction endonucleases Ecl136II and XbaI and the fragment approx. 2120 bp in size with the gap2 gene was purified from a 0.8% agarose gel (QIAquick Gel Extraction Kit der Firma Qiagen, Hilden, Germany). The fragment with the gap2 gene was then ligated with the vector pEC-K18mob2 (T4-Ligase, Roche Diagnostics GmbH, Mannheim; Germany). The ligation batch was transformed in the E. coli strain DH5alphamcr (Hanahan, In: DNA cloning. A Practical Approach. Vol. I, IRL-Press, Oxford, Washington D.C., USA). Selection for plasmid-carrying cells was made by plating out the transformation batch on LB agar (Sambrook et al., Molecular Cloning: A Laboratory Manual. 2^{nd} Ed., Cold Spring Harbor Laboratory Press, Cold Spring Harbor, N.Y., 1989), which had been supplemented with 25 mg/l kanamycin. Plasmid DNA was isolated from a transformant with the aid of the QIAprep Spin Miniprep Kit from Qiagen (Hilden, Germany) and checked by treatment with the restriction enzyme EcoRI with subsequent agarose gel electrophoresis. The plasmid was called pEC-K18mob2gap2exp and is shown in FIG. 2.

### Example 4

Transformation of the Strain DSM5715 with the Plasmid pEC-K18mob2gap2exp

The strain DSM5715 was transformed with the plasmid pEC-K18mob2gap2exp using the electroporation method described by Liebl et al., (FEMS Microbiology Letters, 53:299-303 (1989)). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 25 mg/l kanamycin. Incubation was carried out for 2 days at 33° C.

Plasmid DNA was isolated from a transformant by conventional methods (Peters-Wendisch et al., 1998, Microbiology, 144, 915-927), cleaved with the restriction endonuclease EcoRI, and the plasmid was checked by subsequent agarose gel electrophoresis. The strain obtained in this way was called DSM5715/pEC-K18mob2gap2exp.

### Example 5

Preparation of Lysine

The C. glutamicum strain DSM5715/pEC-K18mob2gap2exp obtained in example 4 was cultured in a nutrient medium suitable for the production of lysine and the lysine content in the culture supernatant was determined.

For this, the strain was first incubated on an agar plate with the corresponding antibiotic (brain-heart agar with kanamycin (25 mg/1)) for 24 hours at 33° C. Starting from this agar plate culture, a preculture was seeded (10 ml medium in a 100 ml conical flask). The complete medium CgIII was used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH was brought to pH 7.4 | | |

Kanamycin (25 mg/l) was added to this. The preculture was incubated for 16 hours at 33° C. at 240 rpm on a shaking machine. A main culture was seeded from this preculture such that the initial OD (660 nm) of the main culture was 0.05. Medium MM was used for the main culture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 100 | g/l |
| (NH₄) ₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state.

Culturing is carried out in a 10 ml volume in a 100 ml conical flask with baffles. Kanamycin (25 mg/l ) was added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection.

The result of the experiment is shown in table 1.

TABLE 1

| | OD | Lysine HCl |
|---|---|---|
| Strain | (660 nm) | g/l |
| DSM5715 | 11.8 | 14.43 |
| DSM5715/pEC-K18mob2gap2exp | 12.1 | 15.21 |

The abbreviations and designations used have the following meaning:

| | |
|---|---|
| Kan: | Resistance gene for kanamycin |
| per: | Gene for control of the number of copies from |
| | PGA1 |
| oriV: | ColE1-similar origin from pMB1 |
| rep: | Plasmid-coded replication region from C. |
| | glutamicum plasmid pGA1 |
| RP4mob: | RP4 mobilization site |
| gap2: | gap2 gene from C. glutamicum |
| EcoRI: | Cleavage site of the restriction enzyme EcoRI |
| HindIII: | Cleavage site of the restriction enzyme HindIII |
| Ecl13II: | Cleavage site of the restriction enzyme |
| Ecl136II | Cleavage site of the restriction enzyme XbaI |
| XbaI: | |

Obviously, numerous modifications and variations on the present invention are possible in light of the above teachings. It is therefore to be understood that within the scope of the appended claims, the invention may be practiced otherwise than as specifically described herein.

What is claimed is:

1. An isolated polynucleotide, which encodes a protein comprising the amino acid sequence of SEQ ID NO:2.

2. The isolated polynucleotide of claim 1, wherein said protein has glyceraldehyde 3-phosphate dehydrogenase 2 activity.

3. A vector comprising the isolated polynucleotide of claim 1.

4. A host cell comprising the isolated polynucleotide of claim 1.

5. The host cell of claim 4, which is a Coryneform bacterium.

6. The host cell of claim 4, wherein said host cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

7. A method for detecting a nucleic acid with at least 70% homology to nucleotide of claim 1, comprising contacting a nucleic acid sample with a probe or primer comprising at least 15 consecutive nucleotides of the nucleotide sequence of claim 1, or at least 15 consecutive nucleotides of the complement thereof.

8. A method for producing a nucleic acid with at least 70% homology to nucleotide of claim 1, comprising contacting a nucleic acid sample with a primer comprising at least 15 consecutive nucleotides of the nucleotide sequence of claim 1, or at least 15 consecutive nucleotides of the complement thereof.

9. A process for screening for polynucleotides, which encode a protein having glyceraldehyde 3-phosphate dehydrogenase 2 activity comprising
a) hybridizing the isolated polynucleotide of claim 1 to the polynucleotide to be screened;
b) expressing the polynucleotide to produce a protein; and
c) detecting the presence or absence of glyceraldehyde 3-phosphate dehydrogenase 2 activity in said protein.

10. A method for making a glyceraldehyde 3-phosphate dehydrogenase 2 protein, comprising culturing the host cell of claim 4 for a time and under conditions suitable for expression of the glyceraldehyde 3-phosphate dehydrogenase 2 protein; and collecting the glyceraldehyde 3-phosphate dehydrogenase 2 protein.

11. An isolated polynucleotide, which comprises SEQ ID NO:1.

12. An isolated polynucleotide, which is complimentary to the polynucleotide of claim 11.

13. An isolated polynucleotide, which is at least 70% identical to the polynucleotide of claim 11.

14. An isolated polynucleotide, which is at least 80% identical to the polynucleotide of claim 11.

15. An isolated polynucleotide, which is at least 90% identical to the polynucleotide of claim 11.

16. An isolated polynucleotide, which comprises at least 15 consecutive nucleotides of the polynucleotide of claim 11.

17. An isolated polynucleotide, which hybridizes under stringent conditions to the polynucleotide of claim 11; wherein said stringent conditions comprise washing in 5×SSC at a temperature from 50 to 68° C.

18. The isolated polynucleotide of claim 11, which encodes a protein having glyceraldehyde 3-phosphate dehydrogenase 2 activity.

19. A vector comprising the isolated polynucleotide of claim 11.

20. A host cell comprising the isolated polynucleotide of claim 11.

21. The host cell of claim 20, which is a Coryneform bacterium.

22. The host cell of claim 20, wherein said host cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

23. A process for screening for polynucleotides, which encode a protein having glyceraldehyde 3-phosphate dehydrogenase 2 activity comprising
a) hybridizing the isolated polynucleotide of claim 11 to the polynucleotide to be screened;
b) expressing the polynucleotide to produce a protein; and
c) detecting the presence or absence of glyceraldehyde 3-phosphate dehydrogenase 2 protein activity in said protein.

24. A method for detecting a nucleic acid with at least 70% homology to nucleotide of claim 11, comprising contacting a nucleic acid sample with a probe or primer comprising at least 15 consecutive nucleotides of the nucleotide sequence of claim 11, or at least 15 consecutive nucleotides of the complement thereof.

25. A method for producing a nucleic acid with at least 70% homology to nucleotide of claim 11, comprising contacting a nucleic acid sample with a primer comprising at least 15 consecutive nucleotides of the nucleotide sequence of claim 11, or at least 15 consecutive nucleotides of the complement thereof.

26. A method for making a glyceraldehyde 3-phosphate dehydrogenase 2 protein, comprising
a) culturing the host cell of claim 20 for a time and under conditions suitable for expression of the glyceraldehyde 3-phosphate dehydrogenase 2 protein; and
b) collecting the glyceraldehyde 3-phosphate dehydrogenase 2 protein.

27. A Coryneform bacterium, which comprises an enhanced gap2 gene.

28. The Coryneform bacterium of claim 27, wherein said gap2 gene comprises the polynucleotide sequence of SEQ ID NO:1.

29. Escherichia coli DSM 14407.

30. A process for producing L-amino acids comprising culturing a bacterial cell in a medium suitable for producing L-amino acids, wherein said bacterial cell comprises an enhanced gap2 gene.

31. The process of claim 30, wherein said bacterial cell is a Coryneform bacterium or Brevibacterium.

32. The process of claim 31, wherein said bacterial cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

33. The process of claim 30, wherein said gap2 gene comprises the polynucleotide sequence of SEQ ID NO:1.

34. The process of claim 30, wherein said L-amino acid is L-lysine.

35. The process of claim 30, wherein said bacteria further comprises at least one gene whose expression is enhanced, wherein said gene is selected from the group consisting of dapA, gap, tp1, pgk, zwf, pyc, mqo, lysC, lysE, hom, ilvA, ilvBN, ilvD and zwa 1.

36. The process of claim 30, wherein said bacteria further comprises at least one gene whose expression is attenuated, wherein said gene is selected from the group consisting of pck, pgi, poxB, and zwa2.

37. An isolated polypeptide comprising the amino acid sequence of SEQ ID NO:2.


SEQUENCE LISTING (4 sequences)

SEQ ID NO: 1 - DNA, 1679 bp, Corynebacterium glutamicum
  CDS (200)..(1639)
agggaactgggattacgcctactgcctggtaggagtcggcctggaatcgattgcgaagggtagtgcaaagcagatactggaatcattaacaccttccgctttgggctaatgttggggggagtgctttcaactatccacgagagctgcccagtgataaaccccgggttaaccccacgcctaagtcagtgaaggactttttatgacgcacaaccacaaggactggaacgatcgcattgcagttgcggaggaaatggtgccgttgatcgggcgcctgcaccgcaacaacaacgtggtggtttccgtattcggtcgtctccttgtgaatgtctcagacatcgatatcatcaagtctcaccgctacgcccgccacatcatatccaaggaacttccactggaaagctccttggatattttgcgcgaactggtagatatgaaccttggtaccgcatcgatcgacctgggacagctggcctacagcttcgaagaatccgaaagcaccgacctgcgtgccttcctggaggacgctctcgcgccggtcattggtgcggaaaccgacatcaacccaactgatatcgtgctgtacggtttcggccgcatcggtcgcctgctggcccgcatcctggtttcccgcgaggcactgtatgacggtgctcgtctgcgcgccatcgtggtccgcaaaaatggtgaagaagacctggtcaagcgcgcatccttgctgcgtcgtgattctgtccacggtggattcgatggcaccatcaccaccgattatgacaacaacatcatctgggccaacggcaccccaatcaaggtcatctactccaatgacccagccaccattgattacaccgaatacggcatcaatgacgccgtcgtggtagacaacaccggccgctggcgtgaccgcgaaggcctgtcccagcacctcaagtccaagggcgttgccaaggttgtactcaccgcgccgggcaagggcgatctgaagaacatcgtgtacggcatcaaccacaccgacatcaccgcagatgatcagatcgtttccgcagcatcatgcaccaccaatgccattaccccagtgctcaaggtgatcaatgatcgctacggcgtggaattcggccacgtagaaaccgttcactccttcaccaatgaccagaacctgatcgacaacttccacaagggttctcgccgtggtcgcgcagcaggtctgaatatggttctcaccgaaaccggcgctgcaaaggctgtatccaaggcgcttccagagctggaaggcaagctcaccggcaatgccatccgcgttcctacccctgacgtgtccatggctgtgctcaacttgaccctgaacacggaggtggaccgcgatgaggtcaacgagttcctccgccgtgtgtccctgcactctgacttgcgccagcaaatcgactggatccgttccccagaggttgtttccactgacttcgtgggcaccacccacgcgggcatcgttgatggtctagccaccatcgcaaccggtcgccacctggtgctttacgtgtggtacgacaacgagttcggctactccaaccaggtcattcgcatcgtcgaggagatcgccggcgtgcgtcctcgcgtgtacccggagcgcaggcagccagccgtactataggttatccaagcctaatacactacgattaggcatatga

SEQ ID NO: 2 - protein, 480 aa, Corynebacterium glutamicum
MTHNHKDWNDRIAVAEEMVPLIGRLHRNNNVVVSVFGRLLVNVSDIDIIKSHRYARHIISKELPLESSLDILRELVDMNLGTASIDLGQLAYSFEESESTDLRAFLEDALAPVIGAETDINPTDIVLYGFGRIGRLLARILVSREALYDGARLRAIVVRKNGEEDLVKRASLLRRDSVHGGFDGTITTDYDNNIIWANGTPIKVIYSNDPATIDYTEYGINDAVVVDNTGRWRDREGLSQHLKSKGVAKVVLTAPGKGDLKNIVYGINHTDITADDQIVSAASCTTNAITPVLKVINDRYGVEFGHVETVHSFTNDQNLIDNFHKGSRRGRAAGLNMVLTETGAAKAVSKALPELEGKLTGNAIRVPTPDVSMAVLNLTLNTEVDRDEVNEFLRRVSLHSDLRQQIDWIRSPEVVSTDFVGTTHAGIVDGLATIATGRHLVLYVWYDNEFGYSNQVIRIVEEIAGVRPRVYPERRQPAVL

SEQ ID NO: 3 - DNA, 20 bp, Corynebacterium glutamicum
ttgaagtggagccggacgag

SEQ ID NO: 4 - DNA, 20 bp, Corynebacterium glutamicum
cctatagtacggctggctgc